(19) United States
(12) Patent Application Publication
(10) Pub. No.: US 2025/0144037 A1
(43) Pub. Date: May 8, 2025

(54) mRNA VACCINE COMPOSITION WITH IMPROVED STORAGE STABILITY

(71) Applicants: Hongik University Industry-Academia Cooperation Foundation, Seoul (KR); LUCA AICELL, INC., Anyang-si (KR); KOREA NATIONAL INSTITUTE OF HEALTH, Cheongjusi (KR)

(72) Inventors: Minsub CHUNG, Seoul (KR); Kyongman AN, Anyang-si (KR); Taehoon KIM, Anyang-si (KR); Minjeong KIM, Seoul (KR); Hyunjin KIM, Seoul (KR); Taekyoung LEE, Seoul (KR); Dokeun KIM, Cheongjusi (KR); Hye Sook JEONG, Cheongjusi (KR); Hyeon Guk KIM, Cheongjusi (KR); Jin-Seung YUN, Cheongjusi (KR)

(73) Assignees: Hongik University Industry-Academia Cooperation Foundation, Seoul (KR); LUCA AICELL, INC., Anyang-si (KR); KOREA NATIONAL INSTITUTE OF HEALTH, Cheongjusi (KR)

(21) Appl. No.: 18/941,843

(22) Filed: Nov. 8, 2024

(30) Foreign Application Priority Data
Nov. 8, 2023 (KR) 10-2023-0153741

(51) Int. Cl. A61K 9/51; A61K 39/00; A61K 47/10

(52) U.S. Cl. CPC A61K 9/5123; A61K 39/0011; A61K 47/10; A61K 2039/53

(57) ABSTRACT

Provided are a mRNA vaccine composition with improved storage stability and a method of improving storage stability of the vaccine composition.

### BACKGROUND

### Technical Field

The present invention relates to an mRNA vaccine composition with improved storage stability.

### Background Art

The global response to COVID-19, which emerged in December 2019, has witnessed the rapid development and deployment of various COVID-19 vaccines worldwide, underscoring the societal urgency of vaccination to shift the pandemic to an endemic state. Notably, mRNA vaccines utilizing Lipid Nanoparticle (LNP) technology, such as BNT162b2 co-developed by BioNTech and Pfizer in Germany, and mRNA-1273 developed by Moderna in the United States, have emerged as frontrunners. These mRNA vaccines offer enhanced adaptability to mutations, coupled with streamlined production processes that have propelled them to receive swift FDA approvals in November 2020, revolutionizing vaccine development speed. However, mRNA vaccines, despite their groundbreaking potential, remain in a stage of refinement as a novel vaccine platform.

A significant limitation of the new vaccine paradigm lies in the necessity for specialized cold chain systems to enable long-term storage. While conventional vaccines can be preserved for over 6 months at temperatures between 2° C. and 8° C., the inherent fragility of mRNA and LNP delivery systems demands storage and transportation at ultralow temperatures. While some virus vector-based vaccines, such as AZD1222 and JNJ-78436735, can maintain efficacy for over a month under refrigeration (2˜8° C.), mRNA vaccines like BNT162b2 and mRNA-1273 require storage at temperatures as low as −90 to −60° C. and −20° C., respectively, to uphold efficacy for a similar duration. The need for such extreme cold conditions, as seen with BNT162b2, poses a challenge for vaccination in healthcare facilities lacking specialized cold storage infrastructure. These issues highlight the significant cost and logistical burdens associated with mRNA vaccine distribution, particularly for resource-limited regions.

Recent studies in the field of lipid-based nanoparticles (LNPs) have revealed promising prospects for the storage and preservation of biologically active molecules, such as small interfering (si)RNA and mRNA. These molecules have gained attention due to their potential therapeutic applications, but their inherent instability poses challenges for long-term storage. To address this issue, recent investigations have focused on the cryoprotectants and lyophilization and of LNPs.

Pengxuan Zhao investigated the long-term storage region of Lipid-like nanoparticles (LLNs) designed for mRNA delivery. By systematically exploring the effects of cryoprotectants on LLNs stability during freezing and lyophilization processes, Zhao et al. successfully identified optimal conditions. Their findings indicated that LLNs containing 5% (w/v) sucrose or trehalose as cryoprotectants, when stored in liquid nitrogen, exhibited sustained mRNA delivery efficiency for a period exceeding 3 months.

While the lyophilization of LNPs presents a promising avenue for preservation, it is imperative to consider the potential impact on functional efficacy. Ball et al. reported that siRNA-LNPs can indeed undergo lyophilization; however, upon reconstitution with water, these LNPs demonstrated diminished efficacy in terms of gene silencing in cell culture. This shows the complexity of maintaining biological activity throughout the preservation process.

Existing studies have mainly explored the stability of mRNA-LNP formulations for lyophilization over time. However, the present invention uniquely aims to optimize a low-temperature decompression concentration method combined with stabilizers to enhance the long-term preservation of mRNA vaccines at controlled low temperatures (2˜8° C.). Leveraging these innovations, the present invention aims to maintain over 90% of initial efficacy while alleviating cold storage requirements. To achieve this, the present invention focus on optimizing a low-temperature decompression concentration method, in conjunction with stabilizer integration, to bolster the extended stability of mRNA vaccines under refrigeration.

Critical to the successful utilization of LNPs as drug delivery systems are a series of constraints. LNP-based vaccines require particle sizes ranging from 10 to 150 nm for intravenous or intramuscular administration, accompanied by a Poly Dispersity Index (PDI) value below 0.3. These physicochemical parameters, including LNP particle size, polydispersity, and encapsulation efficiency (EE %), significantly impact biological performance and must be upheld during the low-temperature decompression concentration process and subsequent storage. The choice of stabilizer type, concentration, low-temperature decompression concentration process parameters, and temperature selection is paramount for successful preservation.

Therefore, the present invention presents a viable solution for transportation and long-term storage temperature challenges in mRNA-LNP vaccine development, signifying a significant stride forward in this critical field.

### DETAILED DESCRIPTION OF THE INVENTION

### Summary

To solve the above problems, the present invention aims to provide an mRNA vaccine composition with improved storage stability.

### Technical Solution

To solve the problems, the present invention provides a vaccine composition with improved storage stability, comprising: mRNA encapsulated within the lipid nanoparticle; and a stabilizer consisting of glycerol.

The vaccine compositions described in the present invention can be used to deliver any mRNA encoding a suitable antigen. mRNA is a type of RNA that carries information from DNA to ribosomes for translation into coded proteins. mRNA can be synthesized by any of several known methods. For example, mRNA according to the present invention can be synthesized via in vitro transcription (IVT). Briefly, IVT is typically performed with a linear or circular DNA template comprising a promoter, a pool of ribonucleotide triphosphates, a buffer system comprising DTT and magnesium ions, and an appropriate RNA polymerase (e.g., T3, T7 or SP6 RNA polymerase), DNAse I, pyrophosphatase, and/or RNAse inhibitor. The exact conditions will vary depending on the specific application.

Lipid nanoparticles (LNPs) are generally known as nano-sized particles composed of a combination of different lipids. Although many different types of lipids may be included in these LNPs, the LNPs of the present invention may be comprised of a combination of, for example, ionizable lipids, phospholipids, sterols, and PEG lipids.

In the present invention, the term “improved stability” refers to the concept of stabilizing the mRNA structure by preventing the collapse or deformation of the mRNA structure inside or outside the cell. In general, RNA is composed of a single strand and has one more hydroxy group than DNA, so its structural stability is low. Therefore, stabilization in the present invention is to prevent structural collapse or degradation of mRNA.

Glycerol is recognized as a non-toxic compound and is widely used as a food additive, and occupies a prominent position as a vaccine adjuvant. Its versatility extends to intravenous administration for intracranial pressure reduction, and it has been granted “generally recognized as safe” (GRAS) classification by the U.S. Food and Drug Administration (FDA). In the present invention, the glycerol may be included in an amount of 10% (w/v) to 30% (w/v), preferably 30% (w/v), but is not limited thereto.

The term “mRNA vaccine” as used herein is novel and offers many advantages over current cell-based vaccines or toxoid vaccines that utilize live, attenuated or killed pathogens. In addition to safety, mRNA vaccines are cost-effective and offer a flexible design platform. Since antigens-encoding mRNA can be induced to induce specific immune responses, they can be applied to the development of mRNA vaccines for a wide range of therapeutic and preventive purposes for various diseases, including infectious diseases and cancer.

The term “vaccine composition” as used herein means a pharmaceutical composition comprising at least one immunologically active component that induces an immunological response in an animal. The immunologically active component of the vaccine may comprise appropriate elements of live or killed virus (subunit vaccines), prepared by destroying whole virus or its growth culture, then prepared by a purification step to obtain the desired structure(s), or by a synthetic process induced by appropriate manipulation of a suitable system such as, but not limited to, bacteria, insects, mammals or other species, followed by isolation and purification, or by induction of the synthetic process in an animal requiring vaccination by direct incorporation of genetic material using an appropriate pharmaceutical composition (polynucleotide vaccination). The vaccine may comprise one or more of the elements described above at the same time.

In one embodiment of the present invention, the vaccine composition may additionally comprise a pharmaceutically acceptable excipient, diluent or carrier. The “pharmaceutically acceptable excipient, diluent or carrier” may mean an excipient, diluent or carrier that does not irritate a living organism and does not inhibit the biological activity or properties of the compound being injected. Wherein, “pharmaceutically acceptable” means that it does not inhibit the activity of the active ingredient and does not have a toxicity greater than that to which the application (prescription) is adapted.

Suitable carriers for vaccines are known to those skilled in the art and include, but are not limited to, proteins, sugars, and the like. The carrier may be an aqueous solution, or a non-aqueous solution, a suspension or an emulsion. Structured or amorphous organic or inorganic polymers can be used as immunoadjuvants to increase immunogenicity. Immunoadjuvants are generally known to play a role in promoting immune responses through chemical and physical binding to antigens. Amorphous aluminum gel, oil emulsion, double oil emulsion, and immunosol can be used as immunoadjuvants. Additionally, various plant-derived saponins, levamisole, CpG dinucleotide, RNA, DNA, LPS, and various types of cytokines can be used to stimulate the immune response. The immune composition can be used as a composition for inducing an optimal immune response by combining various adjuvants and immune response-stimulating additives. Additionally, stabilizers, inactivators, antibiotics, preservatives, etc. may be used as compositions that can be added to the vaccine. Depending on the route of administration of the vaccine, the vaccine antigen can also be mixed with distilled water, buffer solution, etc.

The vaccine composition can be formulated and used in the form of oral formulations such as powders, granules, tablets, capsules, suspensions, emulsions, syrups, aerosols, external preparations, suppositories, or unit dose ampoules or multiple dose injections, respectively, according to conventional methods. When formulating the vaccine composition, it can be prepared by adding diluents or excipients such as commonly used fillers, bulking agents, binders, wetting agents, disintegrating agents, or surfactants.

When the vaccine composition is prepared as a parenteral formulation, it can be formulated into the form of an injection, a transdermal administration agent, a nasal inhaler, and a suppository according to a method known in the art together with a suitable carrier. When formulated as an injection, suitable carriers include sterile water, ethanol, polyols such as glycerol or propylene glycol, or mixtures thereof, and preferably, Ringer's solution, phosphate buffered saline (PBS) containing triethanolamine, sterile water for injection, or an isotonic solution such as 5% dextrose can be used. When formulated as a transdermal agent, it can be formulated in the form of ointment, cream, lotion, gel, external solution, paste, liniment, aerosol, etc. For nasal inhalation, it can be formulated in the form of an aerosol spray using a suitable propellant such as dichlorofluoromethane, trichlorofluoromethane, dichlorotetrafluoroethane, or carbon dioxide, and when formulated as a suppository, the base materials that can be used include witepsol, Tween 61, polyethylene glycol, cocoa butter, laurin butter, polyoxyethylene sorbitan fatty acid ester, polyoxyethylene stearate, and sorbitan fatty acid ester.

The route of administration of the vaccine composition may be through any general route as long as it can reach the target tissue, and specifically, the vaccine composition may be selected from the group consisting of compositions for intramuscular administration, subcutaneous administration, intraperitoneal administration, intravenous administration, oral administration, dermal administration, ocular administration, nasal administration, and intracerebral administration.

The vaccine composition can be administered in a pharmaceutically effective amount, wherein the term “pharmaceutically effective amount” means an amount sufficient to treat or prevent a disease at a reasonable benefit/risk ratio applicable to medical treatment or prevention, and the effective dosage level can be determined depending on the severity of the disease, the activity of the drug, the patient's age, weight, health, sex, the patient's sensitivity to the drug, the time of administration, the route of administration and excretion rate treatment period of the composition of the present invention used, the treatment period, the elements including drugs used in combination or simultaneously with the composition of the present invention used and other elements well known in the medical field. The vaccine composition may be administered alone or in combination with a component known to exhibit a preventive or therapeutic effect against a known COVID-19 omicron virus infection or infectious disease. Taking all of the factors into account, it is important to administer the minimum amount that will achieve the maximum effect without side effects.

The dosage of the vaccine composition can be determined by a person skilled in the art by considering the purpose of use, the degree of toxicity of the disease, the patient's age, weight, sex, medical history, or the type of substance used as an active ingredient. For example, the vaccine composition of the present invention can be administered to an adult at about 0.1 ng to about 1,000 mg/kg, preferably 1 ng to about 100 mg/kg, and the frequency of administration of the composition of the present invention is not particularly limited thereto, but can be administered once a day or administered several times in divided doses. The dosage or frequency of administration does not limit the scope of the present invention in any way.

The vaccine composition can be concentrated using various concentration methods known in the technical field to which the present invention belongs, and specifically, freeze-drying or low-temperature decompression concentration can be used, and preferably, a low-temperature decompression concentration is used, but the invention is not limited thereto.

Also, the low-temperature decompression concentration can be performed at various temperatures, but it is most effective when performed at 4° C.

The mRNA may include various expression constructs that can be used as antigens of the vaccine composition, and specifically may comprise one or more selected from the group consisting of cancer antigens and infectious disease antigens, but is not limited thereto.

The terms “neoplasm,” “cancer,” and/or “tumor,” as used throughout this specification, are not intended to be limited to the types of cancer or tumor that may be exemplified. Therefore, the term encompasses all proliferative disorders such as neoplasms, dysplasias, premalignant or precancerous lesions, abnormal cell growths, benign tumors, malignant tumors, cancers or metastases, wherein the cancers may be selected from the following groups: Leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenal cancer, esophageal cancer, thyroid cancer, blood cancer, and lymphoma. Specific antigens for cancer may be, for example, MelanA/MART1, cancer germline antigen, gp100, tyrosinase, CEA, PSA, Her-2/neu, survivin, and telomerase.

The term “infectious disease” as used throughout the specification is not intended to be limited to the types of infections that may be exemplified herein. Therefore, the term encompasses all infectious agents for which vaccination would be beneficial to the subject. Non-limiting examples include the following virus-induced infections or disorders: Acquired Immunodeficiency Syndrome-Adenoviridae Infections-Alphavirus Infections-Arbovirus Infections-Bell Palsy-Borna Disease-Bunyaviridae Infections-Caliciviridae Infections-Chickenpox-Common Cold-Condyloma Acuminata-Coronaviridae Infections-Coxsackievirus Infections-Cytomegalovirus Infections-Dengue-DNA Virus Infections-Contagious Ecthyma,-Encephalitis-Encephalitis, Arbovirus-Encephalitis, Herpes Simplex-Epstein-Barr Virus Infections-Erythema Infectiosum-Exanthema Subitum-Fatigue Syndrome, Chronic-Hantavirus Infections-Hemorrhagic Fevers, Viral-Hepatitis, Viral, Human-Herpes Labialis-Herpes Simplex-Herpes Zoster-Herpes Zoster Oticus-Herpesviridae Infections-HIV infection-Infectious Mononucleosis-Influenza in Birds-Influenza, Human-Lassa Fever-Measles-Meningitis, Viral-Molluscum Contagiosum-Monkeypox-Mumps-Myelitis-Papillomavirus Infections-Paramyxoviridae Infections-Phlebotomus Fever-Poliomyelitis-Polyomavirus Infections-Postpoliomyelitis Syndrome-Rabies-Respiratory Syncytial Virus Infections-Rift Valley Fever-RNA virus infection-Rubella-Severe Acute Respiratory Syndrome-Slow Virus Diseases-Smallpox-Subacute Sclerosing Panencephalitis-Tick-Borne Diseases-Tumor Virus Infections-Warts-West Nile Fever-Viral disease-Yellow Fever-Zoonoses-etc. Virus-specific antigens may be HIV gag, -tat, -rev or -nef, or hepatitis C antigen; particularly preferred virus-induced infections or disorders are infections of the coronavirus family, such as those caused by coronavirus 229E, coronavirus OC43, SARS-CoV, HCoV NL63, HKU1, MERS CoV or COVID-19. Additional non-limiting examples include the following bacterial- or fungal-induced infections or diseases:

As another example of the present invention, the present invention provides a method for improving the storage stability of a vaccine composition comprising:
a step of preparing lipid nanoparticles encapsulating mRNA;
a step of treating glycerol;
a step of concentrating.

### Effects of the Invention

The present invention relates to an mRNA vaccine composition for improved storage stability, and provides a viable solution for transportation and long-term storage temperature challenges in mRNA-LNP vaccine development by providing a novel mRNA vaccine formulation.

### BRIEF DESCRIPTION OF THE DRAWINGS

FIG. 1 shows a schematic diagram illustrating a comprehensive framework of the present invention, encompassing strategies to enhance the storage stability of an mRNA-LNP vaccine according to one embodiment of the present invention and the analytical methods utilized for monitoring the stability.

FIG. 2 shows the results of low-temperature decompression concentration method and glycerol concentration optimization according to one embodiment of the present invention. A) Schematic diagram for the analysis process for optimization of low-temperature decompression concentration method and glycerol concentration. B) Cell transfection results according to glycerol concentration and when concentrating under low-temperature reduced pressure, C) the amount of solution remaining in the sample according to the evaporation time. Results of the evaporation time optimization experiment by varying the size and PDI by evaporation time: D) glycerol 10%, E) glycerol 30%, F) trehalose, with the size and PDI results by evaporation time, the bar graph shows the particle size and the line graph shows the PDI.

FIG. 3 shows the results of Size and PDI preservation monitoring according to time and temperature of each storage condition according to one embodiment of the present invention. Size: A) 4° C., B) −20° C., C) −80° C. PDI: D) 4° C., E) −20° C., F) −80° C.

FIG. 4 shows the results of HEK-293T cell transfection monitoring according to time and temperature of each storage condition according to one embodiment of the present invention. Luminescence measurement at A) 4° C., B) −20° C., and C) −80° C. And percent of mRNA leakage results at D) 4° C., E) −20° C., and F) −80° C.

### DETAILED DESCRIPTION

Hereinafter, the present invention will be described in more detail through examples. However, the following examples are intended to illustrate the present invention, and the scope of the present invention is not limited to the following examples.

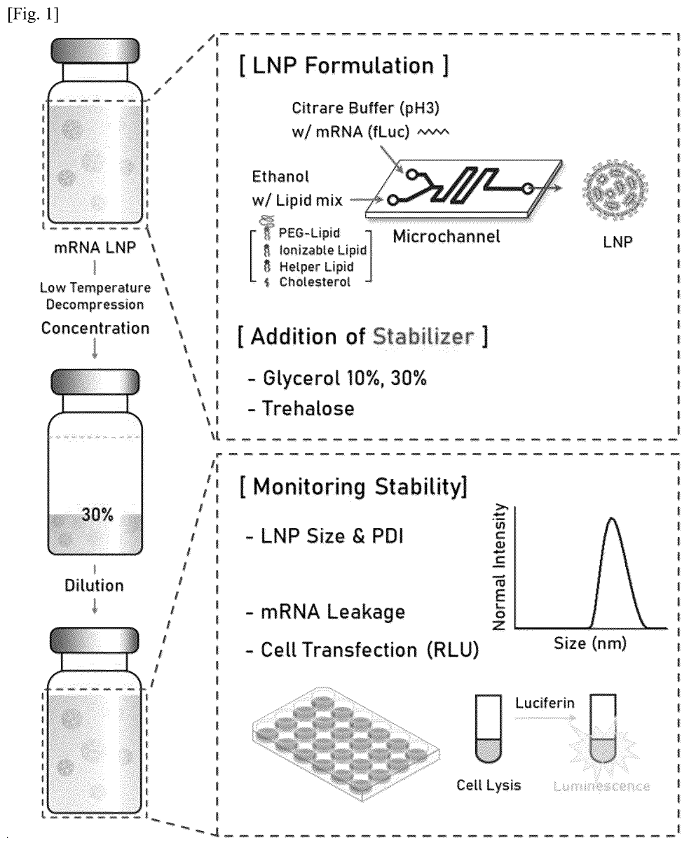

The present invention represents a pivotal advancement in the field of mRNA-LNP vaccine development by enhancing long-term stability under controlled low temperatures (2˜8° C.) through the application of the low-temperature decompression concentration method and stabilizers. By demonstrating that mRNA-LNPs, stabilized by glycerol (10% and 30%) and trehalose, can withstand storage at temperatures ranging from −80°° C. to 4° C. over 6 months, the present invention provides a breakthrough approach to improving vaccine storage stability. Notably, under identical storage conditions, the present invention showcases sustained protein expression of firefly luciferase-encoding mRNA-LNPs in HEK-293T transfection studies. The present invention's process to enhance vaccine storage stability is summarized in FIG. 1.

### <Example 1> Materials

The lipid mix used for LNP formation consists of the following substances and compositions. ((4-hydroxybutyl)azanediyl)bis(hexane-6,1-diyl)bis(2-hexyldecanoate) (ALC-0315, SINOPEG, China), 1,2-dimyristoyl-sn-glycero-3-phosphoethanolamine-N-[methoxy(polyethylene glycol)-2000] (ammonium salt) (14:0 PEG2000 PE, Avanti Polar Lipids Inc, US), 1,2-Distearoyl-sn-glycero-3-phosphocholine (DSPC, Avanti Polar Lipids Inc., US), and Cholesterol (Sigma-Aldrich, US) in a composition of 50:1:10:39. CleanCap® Firefly Luciferase mRNA (CleanCap® FLuc mRNA-(L-7602), TriLink BioTechnologies, US) was used as mRNA for LNP formation. Phosphate-buffered saline (PBS, pH 7.4) was prepared as Na₂H (PO)₄ 8 mM, KH₂(PO)₄ 2 mM, NaCl 137 mM, KCl 2.7 mM, Citrate Buffer as Na₃C₆H₅O₇ 5 mM, Citric acid 5 mM, NaCl 150 mM.

The mRNA was dissolved in the citrate buffer as an aqueous solution. Citrate buffer is prepared by dissolving sodium citrate 5 mM, citric acid 5 mM, and sodium chloride 150 mM in nuclease-free water and adjusting to pH 4.5. It is then sterilized by vacuum filtration using a 0.20 um pore size filter. The concentration of mRNA dissolved in citrate buffer is calculated from the N/P ratio (number of amine groups in cationic lipids (N)/number of phosphate groups in mRNA (P)). By calculating the P value according to the N/P ratio, the amount of mRNA required can be calculated. In the present invention, we used a value of N/P ratio 6.

### <Example 2> mRNA-LNP Formulation

Both the mRNA-LNPs used in the storage experiments and the mRNA-LNPs formed by the microfluidic method were prepared using an NxGen Microfluidics microchannel (The NanoAssemblr® Ignite™, Precision NanoSystems Inc., Canada). The aqueous phase, in which mRNA is dissolved, and the EtOH phase, in which lipids are dissolved, are flowed at a flow rate of 3:1, with 1.5 ml of mRNA solution and 0.5 ml of lipid solution injected. The total flow rate is 12 ml/min. Buffer exchange was performed using an ultra-centrifuging filter (100 kDa pore size) and a Refrigerated, High speed, Microcentrifuge (Micro Centrifuges 1730R, Labogene™, Denmark).

### <Example 3> Low-Temperature Decompression Concentration Method

After forming mRNA-LNP, buffer exchange was performed according to each condition and the stabilizer was mixed. After that, the mRNA-LNP solution with glycerol as stabilizer is concentrated for 2 h, and the mRNA-LNP solution with trehalose as stabilizer is concentrated for 1 h at low temperature (−80° C.) and pressure (5 mTorr), so that the concentration is about 30% of the initial volume.

### <Example 4> Size and PDI Measurements

mRNA-LNP samples were diluted to 40× 2 mL total volume in phosphate buffered saline (PBS), pH 7.4, and transferred into a polystyrene cuvette to measure particle size and polydispersity by DLS (Zetasizer Nano-ZS90, Malvern Instruments, UK), using a refractive index (RI) of 1.590 and an absorption of 0.010 in PBS at 25 C and a viscosity of 0.9073 centipoise (cP) and RI of 1.332. Measurements were made with 10-s run durations with the number of runs automatically determined. Diameters are reported as Z-average.

### <Example 5> mRNA Leakage and Encapsulation Efficiency

RNA encapsulation efficiency and concentration were determined by the Quant-iT RiboGreen Assay (Quant-it™ RiboGreen RNA Kit, Thermo Scientific™, US). Quantification of RNA in LNP formulation was conducted using a standard curve generated from a dilution series of the corresponding fLuc RNA stock. Both standards and samples were diluted with 1× Tris-EDTA (TE) buffer, pH 8.0. Samples were diluted 0.5% (v/v) RNA quantification reagent, 99.5% (v/v) PBS in the polystyrene cuvette. Fluorescence was measured using a spectrofluorophotometer set to 480-nm excitation and 520-nm emission. The standard curve was calculated by linear regression analysis of the fluorescence intensity plotted against the concentration of the standard. RNA encapsulation of LNP samples was determined by comparing the signal of the RNA-binding fluorescent dye RiboGreen in the absence and presence of a detergent (0.1% Triton X-100). In the absence of a detergent, the signal comes only from accessible (unencapsulated) RNA. In the presence of a detergent, the LNP is disrupted so that the measured signal comes from the total RNA (both encapsulated and non-encapsulated).

The encapsulation percentage is calculated using the following equation:

$$Encasulation Efficiency ( \% ) = 1 - \frac{Capsulated mRNA concentration}{Total concentration of mRNA}$$

### <Example 6> Cell Culture and Cell Preparation

The cell line used in the present invention is HEK-293T, a human cell line derived from the HEK-293 cell line that expresses a mutation of the SV40 large T antigen. It is a commonly used cell line for protein expression in biology. The medium used for cell culture is DM (Media is DMEM (low glucose)+F12+10% FBS+1% P/S (antibiotics)), cells were seeded in 25 ml Cell culture flasks (Corning®, US) and cultured in a humidified incubator (Forma™ Direct Heat Incubator, Thermo Scientific™, US) at 37° C., CO₂ 5%. Passage cultures were performed at 3-4 day intervals. 24 hours before transfection, HEK-293T cells were seeded in 24-well plates at a density of 150×10³ cells per well in 500 μl cell culture medium.

### <Example 7> Analyze Luminescence

HEK-293T cells in culture were seeded in 24 wells and 40 ul of the prepared mRNA-LNP sample was injected into the culture medium 24 h later. After 24 h, the cells in each of the 24 wells were removed from the medium and washed with phosphate buffered saline (PBS, pH7.4) to extract the luciferase present inside the cells and measure the luminescence. The cells were then analyzed using the ONE-Glo™ Luciferase Assay System (Promega, US) and a luminometer (GloMax® Navigator Microplate Luminometer, Promega, US).

### <Example 8> Statistical Analysis

All data are presented as mean±standard deviation. Information on the analysis is specified in the figure captions.

### <Test Example 1> Parameter Optimization

The primary objective of the present invention was to achieve prolonged and reliable storage of mRNA-LNP at temperatures exceeding −80° C. through the synergistic employment of a stabilizer and a low-temperature vacuum concentration method. Glycerol and trehalose were selected as the stabilizing agents for the purpose.

Glycerol, recognized as a non-toxic compound, finds widespread application as a food additive and holds a prominent place as a vaccine adjunct. Its versatility extends to intravenous administration for intracranial pressure reduction, and it has secured the classification of “generally recognized as safe (GRAS)” by the US Food and Drug Administration (FDA). Trehalose is utilized as an additive in diverse contexts including ocular solutions and advanced pharmaceutical formulations. In fact, its integration is observed in injectable preparations such as Avastin®, Herceptin® (Roche), Advate® (Baxter), and Lucentis® (Novartis), with Avastin and Lucentis serving as examples of injectable applications. Therefore, optimization of these two stabilizers was performed.

As for the low-temperature decompression concentration method, it was confirmed that complete concentration led to size increase. Consequently, optimization was performed by adjusting the evaporation time to avoid complete concentration.

### <Test Example 2> Optimization of Glycerol Concentration as a Stabilizer

Glycerol and trehalose were used as stabilizers in the drying/concentration process. For glycerol, varying concentrations of 10%, 30%, 50%, and 70% were added to mRNA-LNP formulations in order to find the optimal concentration for use as a stabilizer in the present invention. Experiments were performed to determine concentrations that would not adversely affect cell transfection results.

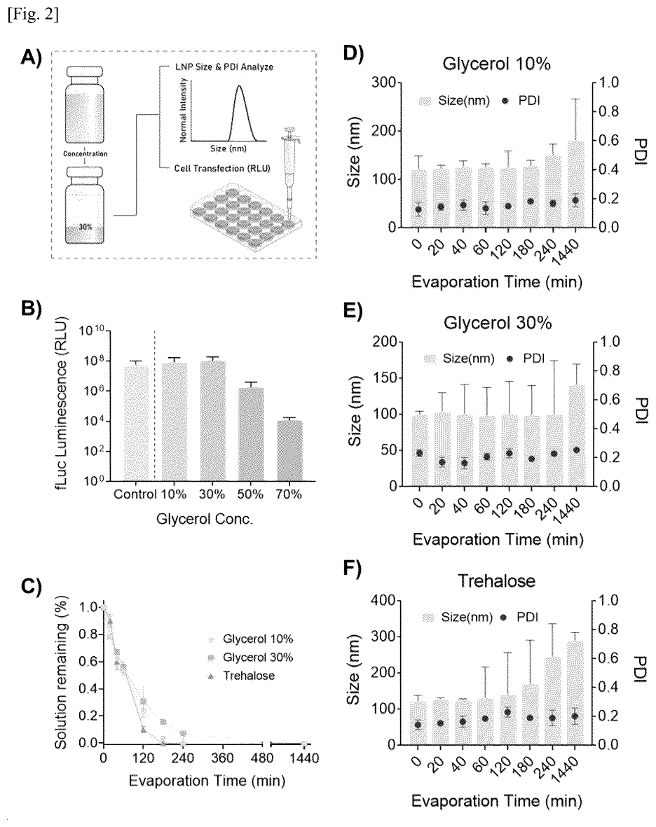

As a result, glycerol concentrations exceeding 50% resulted in a significant decrease in transfection efficacy, which was expected because the viscosity of the solution increased, which interfered with the infection process (FIG. 2b). Considering these results, glycerol concentrations of 10% and 30% were considered suitable for stabilization purposes. In parallel, trehalose was kept constant at 160 mM throughout the present invention.

### <Test Example 3> Optimization of Low-Temperature Decompression Concentration Method

In the case of the low-temperature decompression concentration method, it was confirmed that upon reaching complete concentration, the particle size expanded to approximately 300 nm, which was twice the dimensions of the original particle in FIG. 2. Consequently, a strategy of optimization was implemented, involving adjustments to the evaporation time, aiming to attain maximum concentration without simultaneously increasing size. During this optimization, the particle size and Polydispersity Index (PDI) were measured for each evaporation time from 20 min to 24 hr, across the spectrum of previously selected stabilizer conditions.

As seen in FIG. 2, utilization of 10% and 30% glycerol as stabilizers preserved size and PDI constancy for evaporation time of up to 2 hours. In contrast, for samples containing trehalose as a stabilizer—a substance with relatively lower viscosity than glycerol—the concentration process proceeded quickly. In particular, the particle size and PDI were remained constant during the evaporation time extending up to 1 hour. After concentration, around 40% of the initial sample volume remained in each proper evaporation time seen in FIG. 2c.

### <Test Example 4> Results of mRNA-LNP Stability According to Storage Temperature and Time

Following the treatment of mRNA-LNP with 10% (v/v) glycerol, 30% (v/v) glycerol, and 160 mM trehalose, each formulation was subjected to low-temperature decompression concentration method, in accordance with the optimized concentration times. Subsequently, the concentrated formulations were stored. To assess the impact of time and temperature on preservation, the samples were stored at −80° C., −20° C., and 4° C. Observations for changes in efficacy were recorded at monthly intervals, over 6 months starting immediately after production.

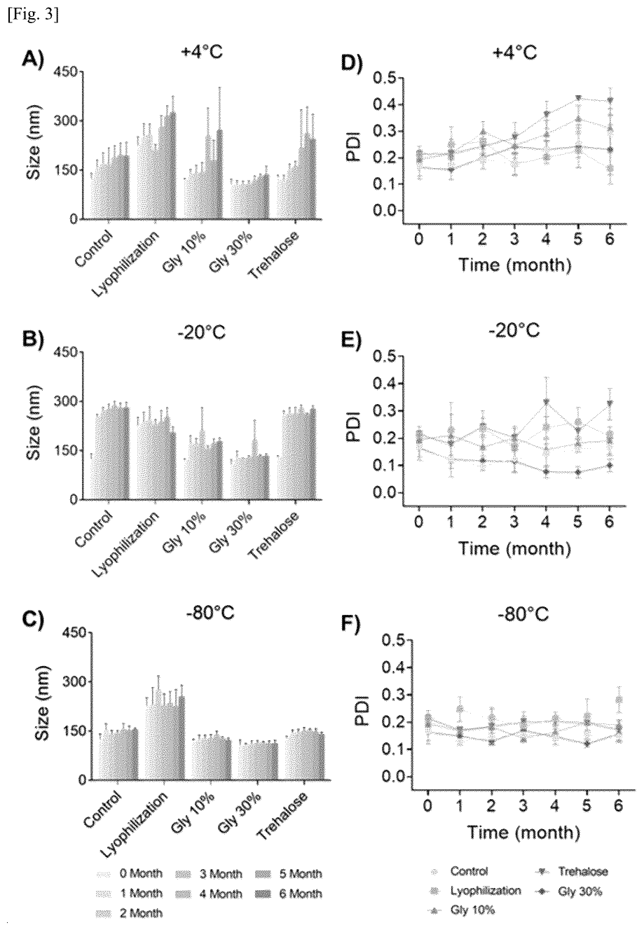

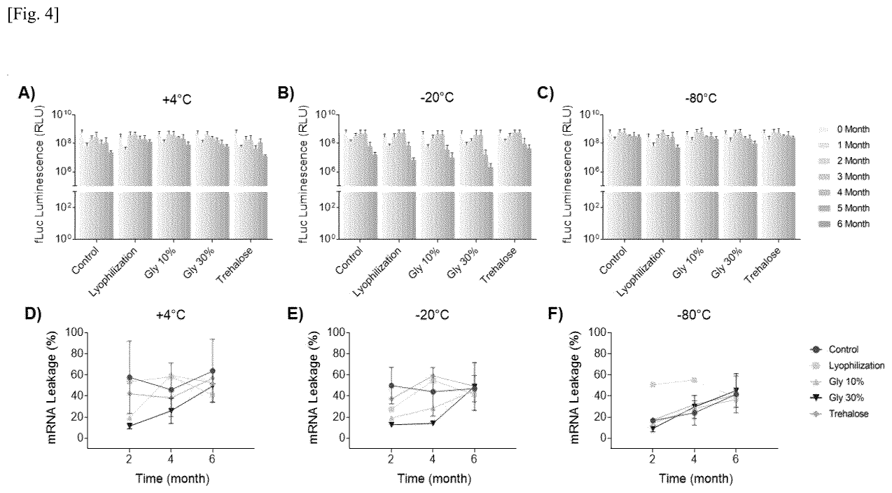

The following 3 analysis methods were used as evaluation measures for changes in efficacy of mRNA-LNP according to time and temperature: 1) Particle size and distribution were determined via Dynamic Light Scattering (DLS) measurements (FIG. 3), The level of luciferase expression after transfection into HEK-293T cells was quantified through luminescence measurements (RLU) using a luminometer and mRNA efflux measurement using technique similar to that employed for encapsulation efficiency determination. (FIG. 4)

These methods collectively provided comprehensive insights into changes in physical properties and efficacy of mRNA-LNP over specified timeframes and storage conditions.

### <Test Example 5> Size and PDI Monitoring Results Over Time

With Results from a 6-month preservation monitoring test, the size and dispersion properties of the particles, determined through DLS, were systematically evaluated in relation to the duration and temperature of mRNA-LNP storage period and temperature. The physical properties of mRNA-LNP for vaccines, including size (less than 150 nm) and PDI value (less than 0.3), were monitored for changes according to each preservation method immediately before storage.

Referring to FIG. 3, it becomes clear that the size of mRNA-LNPs lyophilized without stabilizers showed a large and consistent change in size and PDI across all temperature settings. Moreover, considering temperatures other than −80° C., Pfizer-style LNP control sample and mRNA-LNP preparations containing glycerol 10%, 30% and trehalose as stabilizers show a gradual increase in both size and PDI over time. However, when 30% glycerol is used as the stabilizer, both the size and PDI don't significantly deviate from the initial value over time, regardless of the storage temperature.

### <Test Example 6> Transfection Monitoring Results Over Time

Transfection efficiency was measured to monitor the stability of mRNA-LNPs over time. At FIG. 4, it was confirmed that there was no noticeable decrease to the 12th week under all temperature and stabilizer conditions, but it began to decrease somewhat after the 4th month.

In particular, for samples stored at −20° C., a significant decrease in expression efficiency was observed under all stabilizer conditions, but the decrease was less when trehalose stabilizer was used. In contrast, it can be confirmed that the samples stored at +4° C. showed the greatest decrease in expression efficiency when trehalose as a stabilizer was used and the Pfizer-style LNP, and the samples using glycerol as a stabilizer showed the most stable results. Samples stored at −80° C. appear to be the most stably preserved, with no significant changes under any stabilizer conditions.

### <Test Example 7> Results of Measurement of mRNA Leakage Over Time

The inventors anticipated that the mRNA encapsulated within the lipid nanoparticle (LNP) would gradually escape over time at each storage temperatures of the mRNA-LNP, consequently influencing the transfection. Accordingly, samples stored for 6 months were measured in the same manner as the method for measuring encapsulation efficiency (FIG. 4D, E, F).

In the outcomes of the measurements unveiled that the freeze-drying storage approach resulted in leakage of approximately 50% or more across all temperature conditions, marking it as the most susceptible among the entire set of samples. In particular, when considering the control sample, which was not treated by both stabilizers and the low-temperature decompression concentration method, it was confirmed that at +4° C. and −20° C. storage temperatures, a relatively higher leakage rate was observed compared to the sample stored at −80° C.

Samples that had added stabilizers showed lower mRNA leakage than those that did not, but after 6 months, most samples showed a significant increase in leakage. In particular, when 30% glycerol was used as a stabilizer, the leakage was maintained at less than 15% at all temperatures for 2 months, but increased to more than 40% after 6 months.

### <Test Example 8> Conclusions

In conclusion, the present invention focused on enhancing the storage stability of mRNA vaccines within a controlled low temperature range (2˜8° C.). By fabricating mRNA-LNPs capable of maintaining stable efficacy at refrigeration temperatures, the present invention aims to optimize the low-temperature decompression concentration technique and to identify stabilizing additives through systematic preservation monitoring. The present invention comprises diverse formulations of mRNA-LNPs, comprising those without low-temperature decompression, lyophilization, trehalose-treated followed by low-temperature decompression, and glycerol-treated with low-temperature decompression. Over a six-month period, these formulations were stored at −80° C., −20° C., and 4° C. Through comprehensive analysis encompassing particle size and PDI changes, transfection efficiency in HEK-293T cells, and mRNA leakage monitoring, it is determined that mRNA-LNPs treated with 30% glycerol and subjected to low-temperature decompression exhibited the highest stability across all assessed temperatures, alongside minimal mRNA leakage. This highlights the efficacy of the low-temperature decompression technique at 4° C. in enhancing mRNA vaccine storage stability, with an effective role played by appropriate glycerol concentration as a stabilizer, which can achieve a significant preservation effect when stored at ultra-low temperatures. Moving forward, subsequent in vivo experiments are planned to validate the in vivo toxicity and efficacy of vaccines preserved at low temperatures with stabilizers.

The application of the low-temperature decompression technique and stabilizer in mRNA vaccine development holds potential to alleviate challenges inherent in conventional vaccine transportation and storage in ultra-low temparature, thus mitigating vaccine wastage attributed to refrigeration failures during transportation. This anticipates the prevention of recurrences of the early-stage vaccine storage and transportation issues encountered during the COVID-19 pandemic, as well as alleviating shortage, ensuring economic feasibility, and preventing societal disruptions. The utilization of low-temperature decompression and stabilizers extends beyond COVID-19 vaccines, and offers potential for technical contributions to the realm of mRNA vaccines against other infectious diseases and anticancer agents.

The above description is merely an example of the present invention, and those skilled in the art will appreciate that various modifications may be made without departing from the essential characteristics of the present invention. Accordingly, the embodiments disclosed in this specification are intended to illustrate rather than limit the present invention, and the spirit and scope of the present invention are not limited by these embodiments. The scope of protection of the present invention should be interpreted by the claims below, and all technologies within a scope equivalent thereto should be interpreted as being included in the scope of the rights of the present invention.

What is claimed is:

1. A vaccine composition with improved storage stability, comprising: a mRNA encapsulated within a lipid nanoparticle; and a stabilizer consisting of glycerol.

2. The vaccine composition with improved storage stability, according to claim 1, wherein the glycerol is comprised in an amount of 10% (w/v) to 30% (w/v).

3. The vaccine composition with improved storage stability, according to claim 1, wherein the glycerol is comprised in an amount of 30% (w/v).

4. The vaccine composition with improved storage stability, according to claim 1, wherein the vaccine composition is manufactured by a low-temperature decompression concentration.

5. The vaccine composition with improved storage stability, according to claim 4, wherein the low-temperature decompression concentration is performed at 4° C.

6. The vaccine composition with improved storage stability, according to claim 1, wherein the mRNA comprises one or more selected from the group consisting of cancer antigens and infectious disease antigens.

7. A method of improving storage stability of a vaccine composition comprising:
a step of preparing lipid nanoparticles encapsulating a mRNA;
a step of treating glycerol;
a step of concentrating.

8. The method of improving storage stability of a vaccine composition according to claim 7, wherein the glycerol is comprised in an amount of 10% (w/v) to 30% (w/v).

9. The method of improving storage stability of a vaccine composition according to claim 7, wherein the glycerol is comprised in an amount of 30% (w/v).

10. The method of improving storage stability of a vaccine composition according to claim 7, wherein the concentration process is a low-temperature decompression concentration.

11. The method of improving storage stability of a vaccine composition according to claim 10, wherein the low-temperature decompression concentration is performed at 4° C.

12. The method of improving storage stability of a vaccine composition according to claim 7, wherein the mRNA comprises one or more selected from the group consisting of cancer antigens and infectious disease antigens.
